SEQUENCE LISTING (4 sequences)

SEQ ID NO: 1 - DNA, 34 bp, artificial
  Primer
gttggtacccagtgaggagagtggtgtaaaattg

SEQ ID NO: 2 - DNA, 42 bp, artificial
  Primer
gttgagctccttattatatttttttaacgtttctcaccgatg

SEQ ID NO: 3 - DNA, 40 bp, artificial
  Primer
caacgaaatgcgcaagttccatcatgattatgtcaatatc

SEQ ID NO: 4 - DNA, 40 bp, artificial
  Primer
gatattgacataatcatgatggaacttgcgcatttcgttg

(19) United States
(12) Patent Application Publication
(10) Pub. No.: US 2018/0280321 A1
(43) Pub. Date: Oct. 4, 2018

(54) COMPOSITIONS AND METHODS THAT INHIBIT QUORUM SENSING

(71) Applicant: The Office of Innovation Commercialization, Greensboro, NC (US)

(72) Inventor: Pamela R. Hall, Alburquerque, NM (US)

(21) Appl. No.: 15/557,639

(22) PCT Filed: Mar. 11, 2016
(86) PCT No.: PCT/US2016/021933

§ 371 Date: Sep. 12, 2017

Related U.S. Application Data
(60) Provisional application No. 62/131,928, filed on Mar. 12, 2015.

(51) Int. Cl. A61K 31/122; A61K 31/7028

(52) U.S. Cl. CPC A61K 31/122; A61K 31/7028

(57) ABSTRACT

This disclosure describes pharmaceutical compositions and methods that involve the use of a polyhydroxyanthraquinone to inhibit quorum sensing in a microbe. In some embodiments, the polyhydroxyanthraquinone may be effective to antagonize AgrA function in a microbe. In other embodiments, the polyhydroxyanthraquinone may be effective for prophylactic and/or therapeutic treatment of a skin and soft tissue infection (SSTI) of a subject by a microbe. In still other embodiments, the polyhydroxyanthraquinone may be effective to reduce, limit progression, ameliorate, or resolve, to any extent, a symptom or clinical sign of infection by a microbe.

### CROSS-REFERENCE TO RELATED APPLICATION

This application claims priority to U.S. Provisional Patent Application Ser. No. 62/131,928, filed Mar. 12, 2015, which is incorporated herein by reference.

### SEQUENCE LISTING

This application contains a Sequence Listing electronically submitted to the United States Patent and Trademark Office via EFS-Web as an ASCII text file entitled “2016-03-11-SequenceListing_ST25.txt” having a size of 1 KB and created on Mar. 9, 2016. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, pharmaceutical compositions that include a polyhydroxyanthraquinone. In some embodiments, the composition includes an amount of the polyhydroxyanthraquinone effective to inhibit quorum sensing in a microbe. In other embodiments, the composition includes an amount of the polyhydroxyanthraquinone effective to antagonize AgrA function in a microbe. In other embodiments, the composition includes an amount of the polyhydroxyanthraquinone effective to attenuate a skin and soft tissue infection (SSTI) of a subject by a microbe. In other embodiments, the composition includes an amount of the polyhydroxyanthraquinone effective to limit damage to immune cells of the subject by a microbial virulence factor. In other embodiments, the composition includes an amount of the polyhydroxyanthraquinone effective to limit damage to the tissue by a microbial virulence factor. In other embodiments, the composition includes an amount of the polyhydroxyanthraquinone effective to reduce, limit progression, ameliorate, or resolve, to any extent, a symptoms or clinical sign of infection by a microbe.

In some embodiments, the polyhydroxyanthraquinone can be co-hydroxyemodin (OHM) or an analogue thereof.

In some embodiments, the composition can further include an antimicrobial therapeutic. In some of these embodiments, the antimicrobial therapeutic can be an immunotherapeutic compound. In other embodiments, the antimicrobial therapeutic can be an antibiotic. The antibiotic can be a bacteriocidal antibiotic or bacteriostatic.

In another aspect, this disclosure describes methods of treating a subject having, or at risk of having, an infection by a microbe. Generally, the methods include administering to the subject a composition that includes a polyhydroxyanthraquinone. In some embodiments, the method involves administering an amount of a polyhydroxyanthraquinone effective to inhibit quorum sensing by the microbe. In other embodiments, the method involves administering an amount of a polyhydroxyanthraquinone effective to antagonize AgrA function in the microbe. In other embodiments, the method involves administering an amount of a polyhydroxyanthraquinone effective to attenuate a skin and soft tissue infection (SSTI) of a subject by the microbe. In other embodiments, the method involves administering an amount of the polyhydroxyanthraquinone effective to limit damage to immune cells of the subject by a microbial virulence factor. In other embodiments, the method involves administering an amount of the polyhydroxyanthraquinone effective to limit damage to the tissue by a microbial virulence factor. In still other embodiments, the method involves administering an amount of a polyhydroxyanthraquinone effective to reduce, limit progression, ameliorate, or resolve, to any extent, a symptom or clinical sign of infection by a microbe.

In some embodiments, the microbe may be Staphylococcus aureus. In some of these embodiments, the S. aureus may be methicillin-resistant S. aureus (MSRA).

In some embodiments, the polyhydroxyanthraquinone can be administered prophylactically—i.e., before the subject exhibits a symptom or clinical sign of infection. In other embodiments, the polyhydroxyanthraquinone can be administered therapeutically—i.e., after the subject exhibits a symptom or clinical sign of infection.

In some embodiments, the polyhydroxyanthraquinone can be administered by injection. In other embodiments, the polyhydroxyanthraquinone can be administered by elution from a 30 medical dressing.

In some embodiments, the polyhydroxyanthraquinone can be ω-hydroxyemodin (OHM) or an analogue thereof.

In another aspect, this disclosure describes a method for attenuating virulence of a Staphylococcus spp. Generally, the method includes contacting the Staphylococcus spp. with an amount of a polyhydroxyanthraquinone effective to attenuate virulence of the Staphylococcus spp. In some embodiments, the polyhydroxyanthraquinone attenuates production of alpha-hemolysin. In some embodiments, contacting the polyhydroxyanthraquinone with the Staphylococcus spp. downregulates expression of at least one virulence gene in the Staphylcoccus spp.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG 1. Schematic of the S. aureus accessory gene regulator quorum sensing system and structure of ω-hydroxyemodin. (A) (1) The agr P2 promoter drives expression of the four genes of the operon agrBDCA. (2) AgrD is a pro-peptide which is cyclized to form autoinducing peptide (AIP) and secreted via AgrB. AIP from the four agr alleles vary in length from seven to nine amino acids but all contain a five-membered thiolactone ring. (3) Secreted AIP binds to its cognate receptor AgrC, activating its histidine kinase function leading to phosphorylation of AgrA. (4) AgrA binds to the divergent promoters P2 and P3 as well as the promoters for transcription of the phenol-soluble modulin (PSM) toxins (5). P2 drives a positive-feedback loop resulting in the upregulation of the agr operon, whereas P3 drives transcription of the effector molecule RNAIII. RNAIII leads to the upregulation of virulence factors which contribute to invasive infection. (B) Structure of ω-hydroxyemodin (OHM), molecular mass 286.24.

FIG. 2: ω-Hydroxyemodin inhibits S. aureus quorum sensing by all four agr alleles. (A) Effect of OHM on agr::P3 promoter activation (open symbols) and cell growth (closed symbols), measured by flow cytometry and OD₆₀₀ respectively for agr-I (●), agr-II (▪), agr-III (▴), and agr-IV isolates (♦). (B) Percent cell viability of A549 (●), HEK293 (▪), and HepG2 (▴) cells measured by XTT assay after 24-hour incubation with the indicated concentrations of OHM. Dashed vertical lines indicate concentration used for in vitro assays. Experiments were performed in triplicate or quadruplicate.

FIG. 3: ω-Hydroxyemodin inhibits AgrA binding to promoter DNA. (A) Effect of OHM on agr:P3 promoter activation assessed by rabbit red blood cell lysis for AgrC-WT isolate AH3469 and AgrC-R238H (constitutively active) isolate AH3470. Data are mean relative lysis±SEM compared to vehicle control. Experiments were performed in triplicate. (B) Space-fill model of AgrA_{C} (C-terminal DNA-binding domain) showing potential binding site for OHM. Inset: ball-and-stick representation of OHM binding site. (C) Flow cytometric bead-based assay to determine the effect of OHM on the binding of P2-FAM to biotinylated AgrA_{C} immobilized on streptavidin beads (SA beads). Unlabeled P2 binding to immobilized AgrA_{C} in competition with P2-FAM was included as a specificity control. Data are the mean±SEM (n=3). (D) Surface plasmon resonance analysis of OHM binding to 6-His-tagged AgrA_{C}-C199S immobilized on a nitrilotriacetic acid biosensor. The IC₅₀ was calculated as described in Example [1 or 2?]. ns=not significant, *p<0.05, **p<0.01, ***p<0.001, ****p≤0.0001, by Student's t-test.

FIG. 4: ω-Hydroxyemodin limits abscess formation and dermonecrosis and promotes bacterial clearance in a mouse model of S. aureus SSTI. SKH1 mice were subcutaneously injected with 5-7×10⁷ CFU of LAC or LACΔagr along with OHM (0.2 mg/kg) or vehicle control. (A) Representative images of abscesses and ulcers on day three post-infection (scale bar is 5 mm). (B) Day 3 post-infection abscess, (C) ulcer area and (D) and weight loss of LAC infected mice. (E) Day 3 and day 7 post-infection bacterial burden at the site of infection. Data shown as mean±SEM (LAC, day 3, n=12-16 mice per group; day 7 n=5 mice per group). (F) Δagr day 3 bacterial burden at the site of infection. n=6 mice per group. ns=not significant, *p<0.05, **p<0.01 by Mann-Whitney U test.

FIG. 5: ω-Hydroxyemodin supports immune cell killing of agr+ S. aureus. (A) Mouse macrophage (RAW 264.7) intracellular killing of bacteria pre-treated with OHM or vehicle control. Data are shown as mean±SEM normalized to 100% after one-hour incubation at an MOI of 1:1. n=6 from two independent experiments performed in triplicate. (B) Human polymorphonuclear cell (PMN) intracellular killing of bacteria pre-treated with OHM or vehicle control. Data are the mean±SEM presented as percent survival (top) and Log CFU reduction (bottom) compared to time zero. (C) Supernatant lysis of human PMNs, assessed by LDH release, after a two-hour incubation with sterile supernatant from overnight cultures grown in the presence of OHM or vehicle control. Data are the mean±SEM presented as percent PMN viability compared to 100% lysis by Triton X-100. (B, C) Experiments were performed in triplicate with PMNs from two separate donors. A representative donor experiment is shown. ns=not significant, **p<0.01, ***p<0.001, ****p≤0.0001, by Student's t-test.

FIG. 6: ω-Hydroxyemodin limits pathology and expression of inflammatory cytokines during S. aureus SSTI. (A, Top) Representative hematoxylin-eosin micrographs of 5 μm sagittal sections of day three post-LAC infection abscess tissue. Abscess area is demarcated by fine-dashed line, while ulcer surface length is marked by dash line. (Bottom) Magnification of the transition from normal epithelium to necrotic tissue (left images) and organization of the abscess tissue (right images). (B) Multiplex analysis of cytokines present in the abscess tissue on day three post-infection with LAC or Δagr. (C, E) Quantification of il-1β, tnfα, il-6 and (E) nlrp3 relative to hprt in abscess tissue 24 h post-LAC infection. (D) Western blot analysis and quantification of Hla at the site of infection (day 3) in OHM versus vehicle treated mice (n=4 mice/group). Data are the mean±SEM from infections as described in FIG. 4. *p<0.05, **p<0.01, ***p<0.001, by Student's t-test.

FIG. 7. The purity of OHM was evaluated via a Waters Acquity UPLC system (Waters Corp., Milford, Mass.) using a Waters BEH C18 column (1.7 μm; 2.1×50 mm) and a CH₃CN—H₂O gradient that increased linearly from 20 to 100% CH₃CN over 4.5 minutes. The chromatogram was monitored at 254 nm.

FIG. 8. (A) Quantification of RNAIII, psma and hla by qRT-PCR relative to 16S following a 2 h incubation of USA300 isolate LAC (2×10⁷ CFU/mL) with 50 nM AIP1 and either 5 μg/mL OHM or vehicle control. Data are represented as the fold increase relative to 16S as compared to inoculum bacteria, and normalized to broth with exogenous AIP. (B) Effect of 5 μg/mL OHM on expression of α-hemolysin (Hla) assessed via the rabbit red blood cell lysis assay. HA50 is the bacterial supernatant dilution factor required for lysis of 50% of the RBCs. Data are the mean±SEM of triplicate samples. ns, not significant, **p<0.01, ****p≤0.0001, by Student's t-test.

FIG. 9. (A) Effect of OHM on the electrophoretic mobility shift of the AgrA DNA-binding domain (AgrA_{C}) and P2-FAM complex (‘v’ indicates vehicle control). (B) Effect of OHM on S. epidermidis agr::P3 promoter activation measured by flow cytometry normalized to media control (Broth). Data are the mean±SEM of experiments performed in triplicate. **p<0.01, by Student's t-test.

FIG. 10. Fold change in gene expression in LAC and LACΔagr (Vehicle-treated/OHM-treated) relative to 16S following five hours incubation with 5 μg/mL OHM or vehicle. Dashed line marks two-fold change in transcription compared to vehicle-treated control. ND, not done.

FIG. 11. Effect of OHM pre-treatment of S. aureus on the ability of human polymorphonuclear cells (PMN) to opsonophagocytose bacteria. Data are the mean±SEM, presented as Log CFU of phagocytsosed bacteria at time zero. Experiments were performed in triplicate with PMNs from two separate donors. n.s.=not significant by Student's t-test.

FIG. 12. Therapeutic administration of OHM limits abscess formation and dermonecrosis and promotes bacterial clearance in a mouse model of S. aureus SSTI. SKH1 mice were subcutaneously injected with 7×10⁷ CFU of MRSA isolate LAC. Four hours post-infection, OHM or vehicle control was injected subq near the site of infection. A. Post-infection abscess and B. ulcer area (dermonecrosis). C. Day 7 post-infection bacterial burden at the site of infection. *p<0.05, **p<0.01 by Mann-Whitney U test.

FIG. 13. OHM limits abscess formation and dermonecrosis and promotes bacterial clearance in STZ treated-diabetic mice. Streptozotocin (STZ)-treated, diabetic C57BL/6 mice were purchased from Jackson Laboratories. Mice were subcutaneously infected with 5×10⁷ CFU of MRSA isolate LAC, along with OHM or vehicle control. A. Post-infection abscess and B. ulcer formation (dermonecrosis).

FIG. 14. A single therapeutic dose of OHM plus clindamycin limits abscess formation in a mouse model of S. aureus SSTI. SKH1 mice were subcutaneously injected with 7×10⁷ CFU of MRSA isolate LAC. Four hours post-infection, Vehicle or OHM (5 μg)+/−clindamycin (125 μg) was injected subcutaneously near the site of infection. A. Post-infection abscess area was measured daily. B. Abscess area under the curve comparison. *p<0.05 by Mann-Whitney U test.

FIG. 15. (A) OHM and exemplary analogues. (B) Exemplary OHM analogues.

FIG. 16. Schematic representation of an exemplary hydrogel that solubilizes OHM.

### DETAILED DESCRIPTION OF ILLUSTRATIVE EMBODIMENTS

This disclosure describes w-hydroxyemodin (OHM, formerly also know as citreorosein), a polyhydroxyanthraquinone that can be isolated from, for example, solid-phase cultures of Penicillium restrictum, as a suppressor of quorum sensing. Quorum sensing can control the expression of virulence factors in pathological microbes such as, for example, Staphylococcus aureus. S. aureus is a major cause of invasive skin and soft tissue infections (SSTIs) in both the hospital and community, and is also becoming increasingly antibiotic resistant.

Antibiotic resistant pathogens are a global health threat. Small molecules that inhibit bacterial virulence have been suggested as alternatives and/or adjuncts to conventional antibiotics, as they may limit pathogenesis and increase bacterial susceptibility to host killing. This disclosure describes the use of ω-hydroxyemodin to inhibit quorum sensing in an infectious microbe. While S. aureus was used as an exemplary model infectious microbe, a polyhydroxyanthraquinone may be used to inhibit quorum sensing in other infectious species. At concentrations non-toxic to eukaryotic cells and sub-inhibitory to bacterial growth, OHM prevented agr signaling by all four S. aureus agr alleles. OHM inhibited quorum sensing by direct binding to AgrA, the response regulator encoded by the agr operon, preventing AgrA interaction with the agr promoter DNA. Importantly, OHM was efficacious in a mouse model of S. aureus SSTI. Decreased dermonecrosis with OHM treatment was associated with enhanced bacterial clearance and reductions in inflammatory cytokine transcription and expression at the site of infection. Furthermore, OHM-treatment enhanced immune cell killing of S. aureus in vitro and ex vivo in an agr-dependent manner. These data suggest that bacterial disarmament through suppression of S. aureus quorum sensing may bolster the host innate immune response and limit inflammation.

Many pathogenic bacteria coordinate expression of virulence factors important for invasive infection and pathogenesis through a density-dependent communication system called quorum sensing. Therefore, approaches aimed at disrupting quorum sensing can limit pathogenesis in the host and/or serve as adjuncts to extend the utility of existing antibiotics.

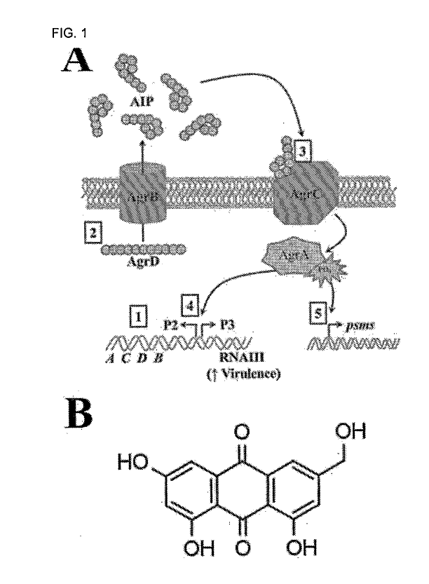

Skin and soft tissue infections (SSTIs) are common infections caused by Staphylococcus aureus. Many of the virulence factors contributing to SSTIs are globally regulated by the accessory gene regulator (agr) (FIG. 1A). The agr system uses a small secreted autoinducing peptide (AIP) to activate a receptor histidine kinase, AgrC, in the bacterial cell membrane. AgrC phosphorylates the transcription factor AgrA, which in turn activates transcription at the P2 and P3 promoters of the operon. P3 activation drives production of the effector of the operon, RNAIII, which regulates expression of over 200 virulence genes that contribute to invasive infection. S. aureus isolates have one of four agr alleles (agr-I, agr-II, agr-III, or agr-IV), each encoding factors that secrete a unique AIP (AIP1, AIP2, AIP3, or AIP4, respectively) that is detected by a cognate AgrC histidine kinase. S. aureus isolates possesses one of the four alleles and an isolate that possesses any one of the four alleles can cause human disease. Disruption of agr-signaling by mutagenesis, monoclonal antibodies, and/or host-factors limits S. aureus infection and reduces pathogenesis.

Polyhydroxyanthraquinones may be isolated from, for example, a culture of the fungus Penicillium restrictum inhibited transcription from all four agr alleles in vitro. Among these, ω-hydroxyemodin (OHM) (FIG. 1B) demonstrated the most potent in vitro agr-I transcription inhibition activity. It was unclear, however, whether the in vitro transcription inhibition activity would result in in vivo inhibition of quorum sensing and/or efficacy in limiting the progression of SSTIs.

This disclosure describes OHM inhibiting in vivo quorum sensing by S. aureus isolates, regardless of which of the four agr alleles possessed by the isolate. Moreover, OHM inhibits quorum sensing at concentrations that are non-cytotoxic for S. aureus or eukaryotic cells. Mechanistically, OHM inhibits agr activation by binding directly to AgrA and blocking binding to agr promoter DNA. In vivo, OHM limits tissue damage and inflammation, and promotes bacterial clearance in a mouse model of S. aureus SSTI. In addition, OHM promotes killing of agr+, but not agr−, S. aureus by both mouse macrophages and human polymorphonuclear cells (PMNs), and limits neutrophil lysis caused by agr-regulated S. aureus secreted virulence factors. This is the first report of a polyhydroxyanthraquinone with in vivo efficacy against S. aureus quorum sensing-dependent virulence.

In addition, these data demonstrate that anti-virulence approaches can limit disease by disarming the bacteria while concurrently bolstering host innate defense. Thus, approaches aimed at augmenting the host response and those aimed at inhibiting bacterial virulence mechanisms provide alternatives to conventional antibiotic therapy that are not mutually exclusive. Because many S. aureus virulence factors antagonize the host innate immune response, inhibiting bacterial virulence can itself augment host defense. Specifically, small molecule-mediated disruption of S. aureus quorum-sensing-dependent virulence can not only limit pathogenesis, but also can reduce inflammation and result in enhanced bacterial clearance.

ω-Hydroxyemodin (OHM) is a Universal Inhibitor of S. aureus agr Transcription

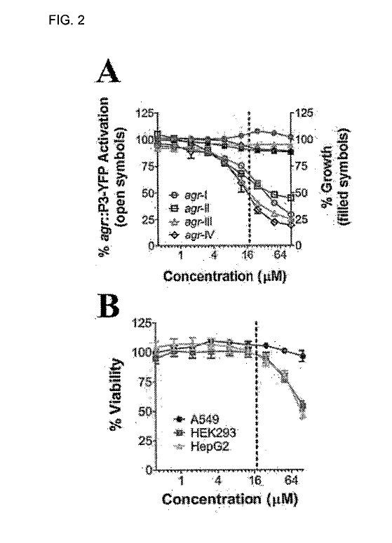

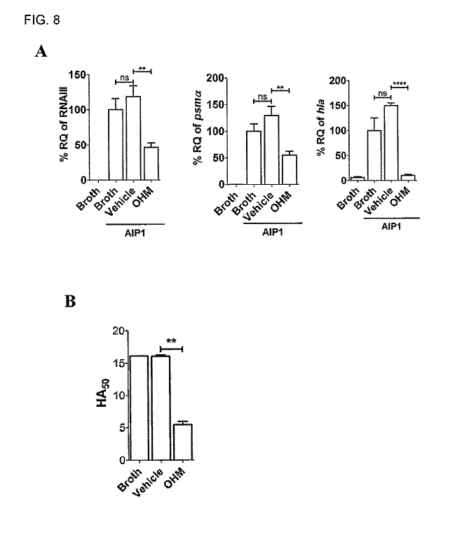

S. aureus isolates having any one of the four agr alleles can contribute to disease in humans. Therefore, a quorum sensing inhibitor that inhibits isolates having any of the agr alleles can provide treatment of a broader spectrum of isolates than a quorum sensing inhibitor effective only against isolates possessing one of a subset of the four alleles. The ability of OHM to inhibit quorum sensing by isolates of all four agr types was assessed using reporter strains expressing yellow fluorescent protein (YFP) under the control of the agr::P3 promoter. OHM inhibited quorum sensing by all four agr types at concentrations that do not impact bacterial growth (FIG. 2A). OHM decreased transcription of the agr effector RNAIII and agr-regulated virulence factors, including phenol soluble modulin alpha (psm α) and alpha-hemolysin (hla) (FIG. 8A). OHM also inhibited production of Hla as demonstrated by red blood cell lysis assay (FIG. 8B). Importantly, at concentrations required for agr-inhibition, OHM was non-toxic to human alveolar (A549), kidney (HEK293), and hepatocyte cell lines (FIG. 2B). Therefore, these data demonstrate that at concentrations non-cytotoxic for eukaryotic cells, OHM is a universal inhibitor of S. aureus agr transcription.

### ω-Hydroxyemodin Antagonizes AgrA Function

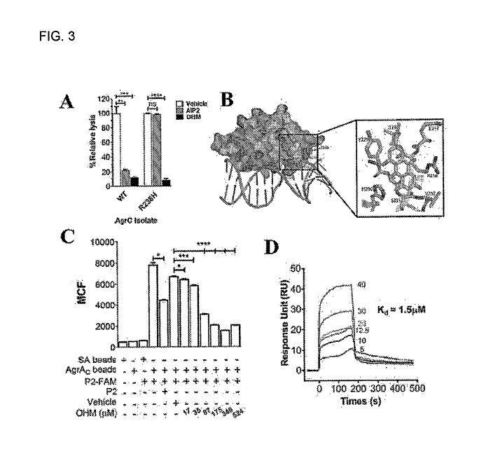

The ability of OHM to inhibit transcription in all four S. aureus agr alleles suggested a target that is well-conserved across all four alleles. To determine whether OHM disrupted AgrC activation, OHM was tested for its ability to inhibit agr-mediated Hla expression determined by lysis of rabbit RBCs, using an agr-I isolate expressing constitutively active AgrC (R238H, (Geisinger et al., 2009, Proc. Natl. Acad. Sci. USA 106:1216-1221)). Inhibitory AIP (AIP2) reduced Hla expression by S. aureus expressing wild-type (WT) but did not inhibit constitutively active AgrC (FIG. 3A). OHM inhibited Hla expression by both isolates (FIG. 3A). These results support a mechanism of action in which OHM inhibits agr-signaling intracellularly, downstream of AgrC activation.

The response regulator, AgrA, functions downstream of AgrC. To further investigate the mechanism of action of OHM, the crystal structure of the C-terminal AgrA DNA binding domain (AgrA_{C}) was evaluated for potential OHM binding sites. The most favorable binding site for OHM was near the AgrA_{C}-DNA interface (FIG. 3B). Docking studies positioned OHM in a pocket between the side chains of H200 and Y229, the latter of which contributes to maximal AgrA activity, and three residues—R218, S231, and V232—that make direct interactions with bound DNA in the AgrA-DNA crystal structure. The crystal structure analysis, the observation that OHM is within hydrogen bonding distance of R218, and that naturally occurring mutations at R218 result in agr-phenotypes, are consistent with OHM inhibiting AgrA binding to promoter DNA.

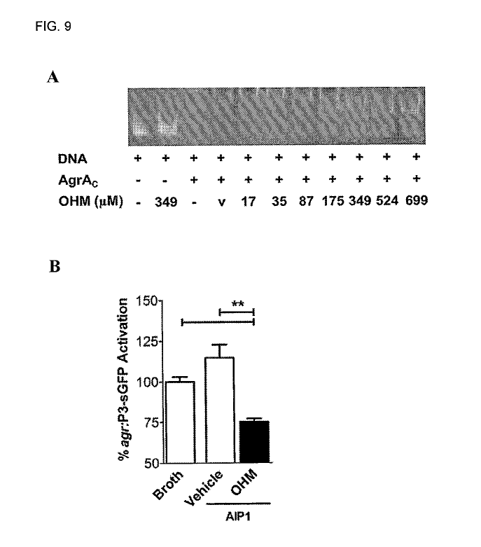

To test whether OHM inhibits AgrA binding to promoter DNA, one can express AgrA_{C} and measure binding to fluorescently labeled duplex agr-promoter DNA encompassing the high affinity binding site located in both agr P2 and P3 promoters (P2-FAM). OHM demonstrated dose-dependent inhibition of AgrA_{C} binding to agr promoter DNA by electrophoretic mobility shift assay (EMSA) (FIG. 9A). In addition, a bead-based assay was used to measure transcription factor binding to target DNA using flow cytometry. Biotinylated AgrA_{C} was immobilized on streptavidin beads (SA beads) and binding to promoter DNA was measured by flow cytometry. OHM again demonstrated dose-dependent inhibition of AgrA_{C} binding to agr promoter DNA (FIG. 3C). Furthermore, OHM bound directly to immobilized AgrA_{C} as shown by surface plasmon resonance (SPR) analysis (FIG. 3D). Together, these data strongly suggest that OHM inhibits agr-signaling by binding to AgrA and blocking AgrA function.

Because the amino acid sequence in the OHM binding site of S. aureus AgrA is highly conserved with that of S. epidermidis AgrA, OHM also can inhibit agr signaling by S. epidermidis. OHM significantly inhibited agr activation by agr-I S. epidermidis (FIG. 9B). AgrA contains a LytTR binding domain, and these domains are used in response regulators of many bacteria/archaea (Nikolskaya et al., 2002, Nucleic Acids Research 30:2453-2459). OHM may inhibit agr signaling in any microbe having a LytTR binding domain with homology to the AgrA LytTR binding domain. Exemplary microbes whose agr signaling may be inhibited by OHM include Gram positive microbes such as, for example, Staphylococcus spp. (e.g., S. lugdunensis, S. pseudintermidedius, and S. saprophyticus), Clostridium spp. (e.g., C. botulinum, C. difficile, and C. perfringens), E. faecalis, L. monocytogenes, Streptococcus spp. (e.g., S. pyogenes, S. pneumonieae, and S. intermedius), Bacillus cereus, and Bacillus subtilis. Gram negative microbes also use LytTRs. Thus, exemplary microbes whose agr signaling may be inhibited by OHM include Gram negative microbes such as, for example, Pseudomonas aeruginosa.

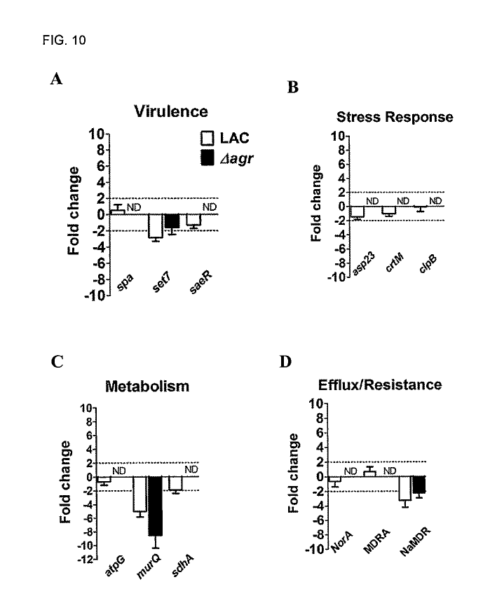

To investigate the specificity of OHM for agr-inhibition, qPCR was used to evaluate the effects of OHM on transcription of a series of agr- and non-agr-regulated genes involved in virulence, the stress response, metabolism and drug efflux and resistance (Table 1, FIG. 10).

TABLE 1
Transcriptional analysis of the agr specificity of OHM

| | | agr- | Fold change in gene expression* | | | |
|---|---|---|---|---|---|---|
| | | regulation/ | (Vehicle/OHM treatment) | | | |
| Focus | Gene | association | LAC | p value | Δagr^{#} | p value |
| Virulence | spa (SAUSA300_0113) | neg | <2 | | ND | |
| | set7 (SAUSA300_0396) | n/a | −2.84 | 0.0019 | <2 | |
| | saeR (SAUSA300_0691) | pos | <2 | | ND | |
| Strass | asp23 (SAUSA300_2142) | n/a | <2 | | ND | |
| response | crtM (SAUSA300_2499) | n/a | <2 | | ND | |
| | clpB (SAUSA300_0877) | n/a | <2 | | ND | |
| Metabolism | atpG (SAUSA300_2059) | n/a | <2 | | ND | |
| | murQ (SAUSA300_0193) | pos | −4.99 | <0.0001 | −8.551 | <0.0001 |
| | sdhA (SAUSA300_1047) | n/a | <2 | | ND | |
| Efflux/ | norA (SAUSA300_0680) | n/a | <2 | | ND | |
| antibiotic | mdrA (SAUSA300_2299) | n/a | <2 | | ND | |
| resistance | NaMDR (SAUSA300_0335) | n/a | −3.23 | 0.0015 | −2.26 | 0.0076 |

*Values are shown if ≥2-fold
^{#}Assay performed with Δagr if ≥ two fold difference with OHM treatment or LAC (ND, not done).

With respect to virulence genes, OHM treatment resulted in a slight increase in transcription of spa, which encodes Protein A and which is negatively regulated by agr. In contrast, expression of the enterotoxin gene set7 decreased with OHM in LAC but not LACΔagr, and OHM had no effect on expression of saeR component of the SaeRS virulence regulator. Likewise, transcription of genes involved in the stress response (asp23, crtM and clpB) was not altered by OHM, suggesting that OHM does not induce a general stress response in LAC under the conditions tested. Among the metabolism genes examined, OHM had no significant effect on transcription of genes involved in electron transport (atpG, sdhA). However, OHM treatment significantly decreased transcription of murQ, an N-acetylmuramic acid 6-phosphate lysase, in both LAC and LACΔagr. Although this protein, which is involved in cell wall recycling, is dispensable for growth in E. coli, its contribution to the growth of Gram positive pathogens is less clear. However, the absence of bactericidal or bacteriostatic effects with OHM treatment suggests that MurQ is not required for growth under the conditions tested. In addition, OHM treatment did not increase transcription of genes examined with potential to contribute to drug efflux or resistance. Therefore, although there are some non-agr effects, these results suggest that OHM is not a general inhibitor of transcription or energetics, or a general inducer of drug efflux. Furthermore, together with the above demonstrations of (i) OHM-mediated agr-inhibition in a whole cell assay, (ii) OHM-mediated inhibition of AgrA_{C} binding to agr promoter DNA by both EMSA and bead-based assay and (iii) direct binding of OHM to AgrA_{C} shown by SPR (FIGS. 2, 3), these results are consistent with a mechanism whereby OHM predominantly functions as an inhibitor of agr-activation.
ω-Hydroxyemodin Attenuates S. aureus SSTI

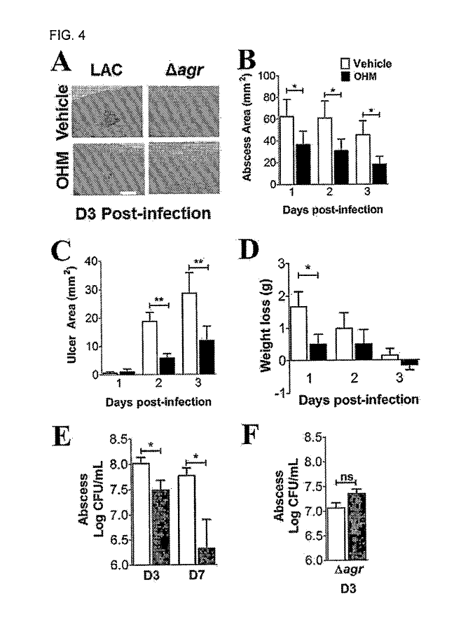

Next, the efficacy of OHM was assessed in an established in vivo mouse model of S. aureus SSTI (Malachowa et al., 2013, Methods Mol. Biol. 1031:109-116). Over the course of a three day infection with USA300 isolate LAC, a single 5 μg dose (0.25 mg/kg) of OHM administered at the time of infection significantly inhibited abscess (FIG. 4A, B) and ulcer (dermonecrosis) formation (FIG. 4A, C), as well as day one post-infection morbidity (assessed by weight loss) compared to vehicle treated controls (FIG. 4D). In contrast, no differences were observed between OHM and vehicle treated mice infected with LACΔagr (FIG. 4A), demonstrating the specificity of OHM for disrupting agr-signaling without directly impacting the host. Importantly, the single OHM treatment reduced day three and day seven post-infection bacterial burden at the site of infection in LAC infected (FIG. 4E), but not Δagr infected mice (FIG. 4F), indicating that mice were better able to combat the infection in the absence of agr-signaling.

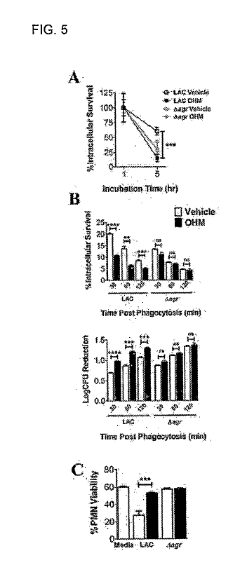

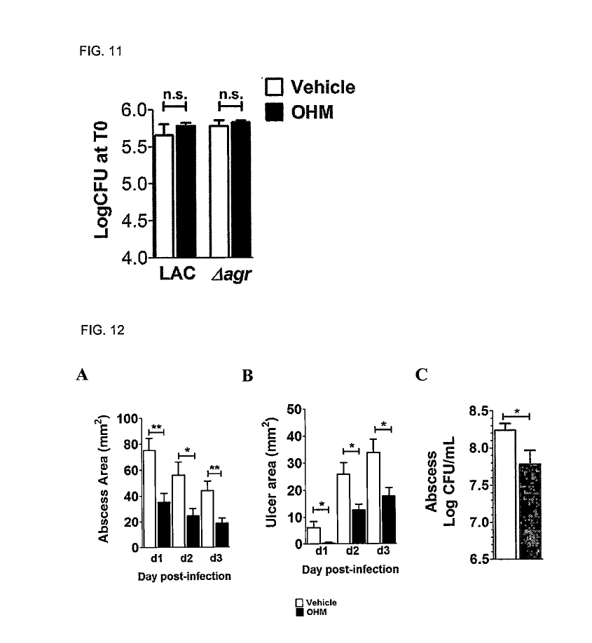

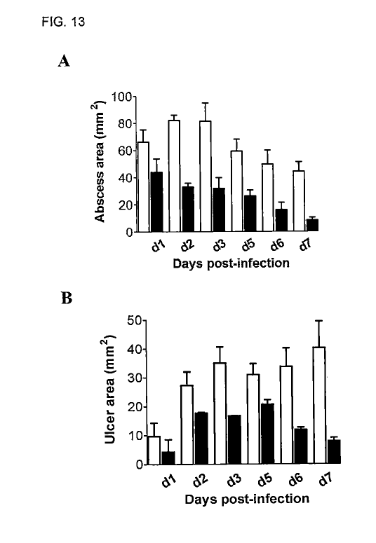

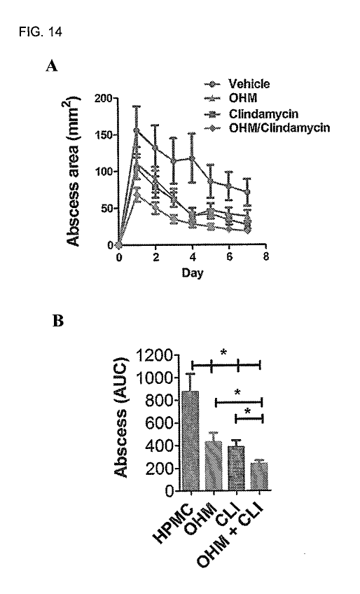

Since OHM treatment supported host-mediated clearance by disrupting agr-signaling, then OHM-treated LAC, but not LACΔagr, can be more readily killed by innate immune cells in vitro compared to vehicle-treated controls. FIG. 5 shows that OHM treatment of LAC, but not LACΔagr, resulted in significantly increased intracellular killing by both mouse macrophages (FIG. 5A) and human PMNs (FIG. 5B) compared to vehicle treated controls. This increased killing was not a result of OHM-mediated effects on opsonophagocytosis, as the total number of bacteria phagocytosed (FIG. 11), and the percent of bacteria phagocytosed relative to the total inoculum, were equivalent regardless of whether the bacteria were pre-treated with vehicle or OHM. Furthermore, OHM-treatment of LAC, but not LACΔagr, protected human PMNs from killing by secreted agr-regulated virulence factors. PMNs showed significantly increased survival in the presence of supernatant from OHM—versus vehicle-treated LAC (FIG. 5C). Together, these results demonstrate that OHM supports host mediated clearance of S. aureus by inhibiting agr-mediated virulence.

ω-Hydroxyemodin Limits Inflammation Mediated by S. aureus Quorum Sensing

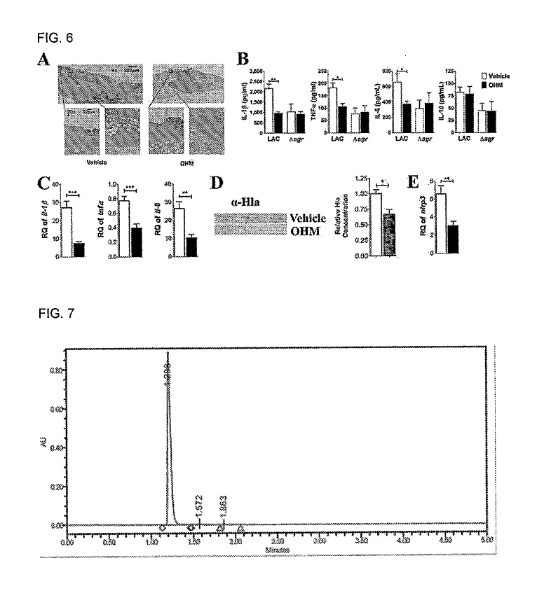

S. aureus uses a variety of virulence factors, many of which are regulated by the agr system, to evade host clearance mechanisms. These virulence factors can cause tissue damage, inflammation, and/or facilitate invasive infection. Therefore, in addition to reducing bacterial burden in LAC infected mice, OHM treatment may result in reducing tissue damage and/or reducing local inflammatory cytokine production compared to vehicle-treated controls. Histological analysis of day three post-infection skin sections confirmed the overall reduction in abscess formation and ulceration in OHM-treated mice (FIG. 6A). Additionally, skin sections from vehicle treated mice displayed a disorganized architecture at both the epithelium to necrosis transition (FIG. 6A, left inset) and at the abscess periphery (right inset) compared to sections from OHM treated mice. OHM treatment resulted in a local cytokine profile matching that of LACΔagr infected mice on day three post-infection (FIG. 6B), with significant reductions in IL-1β, TNFα, and IL-6, but not the anti-inflammatory cytokine IL-10, compared to vehicle treated controls. LAC infected mice treated with OHM also showed reduced transcription of il-1β, tnfα and il-6, at 24 hours post-infection compared to vehicle-treated mice (FIG. 6C). Finally, activation of the NLRP3 inflammasome and subsequent release of IL-1β is induced by pore formation in host cell membranes by Hla, and passive transfer of Hla neutralizing antibodies is sufficient to limit secretion of IL-1β. OHM treated mice showed decreased local Hla expression and decreased transcription of nlrp3 compared to vehicle treated controls (FIG. 6D, E). Together, these data demonstrate that OHM inhibition of agr-signaling limits host tissue damage and inflammation during S. aureus SSTI.

Thus, this disclosure describes compositions that include a polyhydroxyanthraquinone (e.g., ω-hydroxyemodin, OHM, or an analogue thereof) in an amount effective to inhibit quorum sensing in a microbe. While described herein in the context of an exemplary embodiment in which the microbe is S. aureus, the compositions and methods described herein can involve quorum sensing in any microbe having a LytTR binding domain with homology to the AgrA LytTR binding domain. Thus, in some embodiments, OHM may inhibit quorum sensing in Gram positive microbes such as, for example, Staphylococcus spp. (e.g., S. aureus, S. lugdunensis, S. pseudointermedius, and S. saprophyticus), Clostridium spp. (e.g., C. botulinum, C. difficile, and C. perfringens), E. faecalis, L. monocytogenes, Streptococcus spp. (e.g., S. pyogenes, S. pneumoniae, and S. intermedius) and Bacillus subtilis. In other embodiments, OHM may inhibit quorum sensing in Gram negative microbes such as, for example, Pseudomonas aeruginosa.

Also, while described herein in the context of an exemplary embodiment in which the polyhydroxyanthraquinone is OHM (1,3,8-trihydroxy-6-(hydroxymethyl)anthracene-9,10-dione), the compositions and methods described herein can involve any suitable polyhydroxyanthraquinone. Exemplary alternative polyhydroxyanthraquinones include, for example, emodin (1,3,8-trihydroxy-6-methylanthracene-9,10-dion), 2-chloroemodic acid (6-chloro-4,5,7-trihydroxy-9,10-dioxo-9,10-dihydroanthracene-2-carboxylic acid), 2-hydroxyemodic acid (4,5,6,7-tetrahydroxy-9,10-dioxo-9,10-dihydroanthracene-2-carboxylic acid), (+)-2′S-isorhodoptilometrin ((S)-1,3,8-trihydroxy-6-(2-hydroxypropyl)anthracene-9,10-dione), 1′-hydroxy-2′-ketoisorhodoptilometrin (1,3,8-trihydroxy-6-(1-hydroxy-2-oxopropyl)anthracene-9,10-dione), 1′-hydroxyisorhodoptilometrin (3-((1S,2R)-1,2-dihydroxypropyl)-1,6,8-trihydroxyanthracene-9,10-dione), emodic acid (4,5,7-trihydroxy-9,10-dioxo-9,10-dihydroanthracene-2-carboxylic acid), aloe-emodin (1,8-dihydroxy-3-(hydroxymethyl)-9,10-anthracenedione), desmethyl dermoquinone (1,3,8-trihydroxy-6-(2-oxopropyl)anthracene-9,10-dione), and physcion (1,8-dihydroxy-6-methoxy-3-methyl-anthracene-9,10-dione). Moreover, emodin-like structures can exist in dimeric states as sennosides, which also may inhibit quorum sensing as described herein.

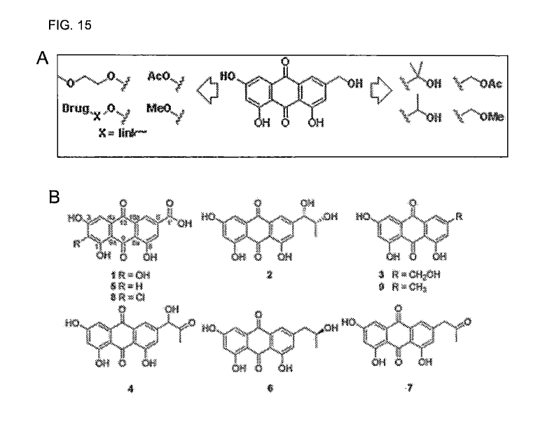

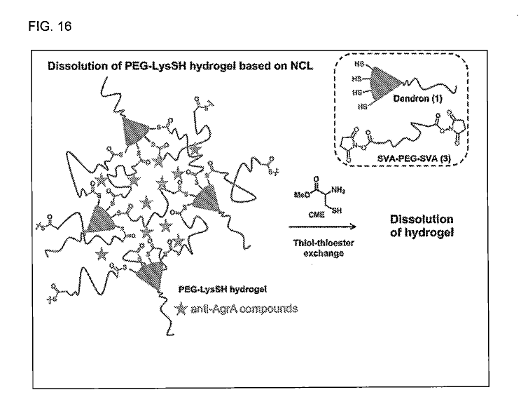

The polyhydroxyanthraquinone also can include an analogue of OHM. Exemplary analogues are illustrated in FIG. 15. In particular, certain analogues can include a drug covalently linked to the OHM core structure as shown. Other analogues may exhibit increased solubility and/or stability compared to OHM. Substitutions may be made in the OHM core structure at, for example, the isolated phenol and/or the primary alcohol. Both of these groups can be reactive as nucleophiles, and the primary alcohol can be oxidized and modified as needed.

The chemical modifications of OHM can involve alkylating or esterifying the phenol group or the primary alcohol (FIG. 15). Although there are two other phenols in the molecule, the targeted phenol is more sterically accessible than the others and is not involved in hydrogen bonding, which is present with the other phenols. Further evidence in support of the selective reactivity of the isolated phenol is that the analogous phenol in emodin is sufficiently more reactive than the other phenols. The primary alcohol, although not as acidic as the phenol, is still sufficiently reactive to be alkylated or acylated. Chemical modification of the OHM core structure may make use of protecting groups when needed.

Such a composition may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with a polyhydroxyanthraquinone without causing any undesirable biological effects or interacting in a deleterious manner with any of the other components of the pharmaceutical composition in which it is contained.

A polyhydroxyanthraquinone may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A pharmaceutical composition also can be administered via a sustained or delayed release, and/or be eluted combined or eluted from a medical dressing such as, for example, a bandage.

For example, FIG. 16 illustrates an exemplary PEG-LysSH hydrogel that can solubilize and provide sustained delivery of a polyhydroxyanthraquinone. A PEG-LysSH hydrogel dressing can provide sustained delivery of the polyhydroxyanthraquinone—either with or without a conventional antimicrobial therapeutic—to the wound site, absorb wound exudates, and/or maintain a moist environment (FIG. 16). The gel can be removed painlessly by, for example, dissolution using an aqueous cysteine solution (a thiol-thiolester exchange mechanism). The release profile of the polyhydroxyanthraquinone be determined under sink conditions (10% fetal bovine serum solution, 1 μg/mL maximum concentration) by HPLC, UV-vis, or MS.

A formulation may be conveniently presented in unit dosage form and may be prepared by methods well known in the art of pharmacy. Methods of preparing a composition with a pharmaceutically acceptable carrier include the step of bringing the polyhydroxyanthraquinone into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

A polyhydroxyanthraquinone may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

The amount of polyhydroxyanthraquinone administered can vary depending on various factors including, but not limited to, the specific polyhydroxyanthraquinone in the composition, the specific microbe for which treatment is needed, the weight, physical condition, and/or age of the subject, and/or the route of administration. Thus, the absolute weight of polyhydroxyanthraquinone included in a given unit dosage form can vary widely, and depends upon factors such as the polyhydroxyanthraquinone used, the species of infectious microbe, age, weight and physical condition of the subject, and/or method of administration. Accordingly, it is not practical to set forth generally the amount that constitutes an amount of polyhydroxyanthraquinone effective for all possible applications. Those of ordinary skill in the art, however, can readily determine the appropriate amount with due consideration of such factors.

In some embodiments, the method can include administering sufficient polyhydroxyanthraquinone to provide a dose of, for example, from about 100 ng/kg to about 50 mg/kg to the subject, although in some embodiments the methods may be performed by administering a polyhydroxyanthraquinone in a dose outside this range. In some of these embodiments, the method includes administering sufficient polyhydroxyanthraquinone to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m²=(wt kg^{0.425}×height cm^{0.725})×0.007184.

In some embodiments, the method can include administering sufficient OHM to provide a dose of, for example, from about 0.01 mg/m² to about 10 mg/m².

In some embodiments, a polyhydroxyanthraquinone may be administered, for example, from a single dose to multiple doses per day, although in some embodiments the method can be performed by administering the polyhydroxyanthraquinone at a frequency outside this range. In certain embodiments, a polyhydroxyanthraquinone may be administered from about once per month to multiple times per day. In other embodiments, a polyhydroxyanthraquinone may be administered on an as needed basis.

A polyhydroxyanthraquinone may be administered to a subject before or after the subject manifests a symptom or clinical sign of infection by a microbe. “Symptom” refers to any subjective evidence of disease or of a patient's condition. “Sign” or “clinical sign” refers to an objective physical finding relating to a particular condition capable of being found by one other than the patient. Treatment that is initiated before a subject manifests a symptom or clinical sign of infection can be considered prophylactic treatment of a subject “at risk” of infection by the microbe. As used herein, the term “at risk” refers to a subject that may or may not actually possess the described risk. Thus, for example, a subject “at risk” of infectious condition is a subject present in an area where other individuals have been identified as having the infectious condition and/or is likely to be exposed to the infectious agent even if the subject has not yet manifested any detectable indication of infection by the microbe and regardless of whether the subject may harbor a subclinical amount of the microbe.

Accordingly, administration of a composition can be performed before, during, or after the subject first exhibits a symptom or clinical sign of the condition or, alternatively, before, during, or after the subject first comes in contact with the infectious agent. Treatment initiated before the subject first exhibits a symptom or clinical sign associated with the condition may result in decreasing the likelihood that the subject experiences clinical evidence of the condition compared to a subject to which the composition is not administered, decreasing the severity of symptoms and/or clinical signs of the condition, and/or completely resolving the condition. Treatment initiated after the subject first exhibits a symptom or clinical sign associated with the condition can be considered therapeutic treatment of the subject, and may result in decreasing the severity of symptoms and/or clinical signs of the condition compared to a subject to which the composition is not administered, and/or completely resolving the condition.

Thus, the method includes administering an effective amount of the composition to a subject having, or at risk of having, a particular condition. In this aspect, an “effective amount” is an amount effective to reduce, limit progression, ameliorate, or resolve, to any extent, a symptom or clinical sign related to the condition.

In some embodiments, the compositions and methods involving the use of a polyhydroxyanthraquinone can be combined with conventional antimicrobial therapies such as, for example, antibiotics or immunotherapies. Thus, a composition as described above can include a polyhydroxyanthraquinone and an antimicrobial therapeutic such as, for example, an antibiotic. The antibiotic may be a bactericidal antibiotic, a bacteristatic antibiotic, and/or a combination of two or more antibiotics. Exemplary antibiotics include, for example, a lincosamide (e.g., lincomycin or clindamycin), a penicillin (e.g., nafcillin), a cephalosporin (e.g., ceftaroline, ceftazidime (alone or in combination with avibactam), or ceftolozane (alone or in combination with tazobactam), a glycopeptide (e.g., vancomycin, oritavancin, dalbavancin), a lipopeptide (e.g., daptomycin), an aminoglycoside (e.g., gentamicin), an oxazolidinones (e.g., linezolid, tedizolid, posizolid, or cycloserine), or a tetracycline (e.g., doxycycline). Exemplary antimicrobial therapeutics include, for example, an immunotherapeutic such as, for example, an antimicrobial antibody treatment and/or antimicrobial cytokine treatment.

The methods described herein can therefore include co-administering a polyhydroxyanthraquinone and an antimicrobial therapeutic. As used herein “co-administering” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another.

Two strategic approaches for reducing the extent and/or likelihood of microbes developing resistance to antibiotics include (i) anti-virulence strategies to disarm bacteria to reduce pathogenesis and (ii) approaches to harness the host immune system to better combat infections. This disclosure describes the use of OHM, an exemplary polyhydroxyanthraquinone that is a natural product isolated from the fungus Penicillium restricnam to address both approaches by directly inhibiting S. aureus quorum-sensing-dependent virulence, while indirectly bolstering the host immune response against S. aureus infection. In a mouse model of S. aureus SSTI, OHM significantly decreases abscess and ulcer formation and promotes bacterial clearance. OHM treatment reduces tissue damage and limits local pro-inflammatory cytokine production to levels seen in mice infected with the agr-deletion mutant. Furthermore, OHM treatment enhances immune cell-mediated killing of S. aureus in an agr-dependent manner. Therefore, these data demonstrate that anti-virulence strategies can limit disease by disarming the bacteria while concurrently reducing inflammation and promoting host innate defense. In addition, this is the first polyhydroxyanthraquinone described with in vivo efficacy against MRSA infection.

Thus, polyhydroxyanthraquinones can be a useful treatment, either prophylactically or therapeutically, for microbial infection. The use of polyhydroxyanthraquinones can provide stand-alone treatment or may be used in conjunction with conventional antimicrobial therapies such as, for example, the use of antibiotics or immunotherapies. While exemplified in the context of skin and soft tissue infections, polyhydroxyanthraquinones can provide anti-bacterial therapy in other applications such as, for example, skin infections associated with diabetes, wound and surgical site infections, ophthalmitis, and pneumonia.

OHM was used in a prophylactic administration model, similar to that previously reported for administration of competing AIP or passive transfer of monoclonal antibodies targeting AIP4 (Park et al., 2007, Chem. Biol. 14:1119-1127; Wright et al., 2005, Proc. Natl. Acad. Sci. USA 102:1691-1696), to demonstrate that small molecule-mediated disruption of agr-signaling in vivo results in an “agr-null-like” host inflammatory profile, as was shown for savirin (Sully et al., 2014, PLoS Pathogens June 12; 10(6):e1004174).

The molecular modeling studies described above positioned OHM near R218 of S. aureus AgrA. This residue, which is strictly conserved across multiple staphylococcal species, is required for agr-function and contributes to AgrA binding to agr promoter DNA. Although the potential exists for OHM to drive selection for an alternative amino acid at residue 218, any such mutation would likely result in agr dysfunction. Selection for quorum sensing deficient isolates is unlikely to be of significant benefit to the pathogen, as these isolates are severely attenuated, more readily cleared by host defenses, and less effective at initiating infection.

The contribution of agr to S. aureus pathogenesis has largely been demonstrated in models of SSTI and pneumonia. While SSTIs frequently result from S. aureus infections, this pathogen causes a variety of disease manifestations, including pneumonia, osteomyelitis, endocarditis and bloodstream infections (BSI). In particular, agr-dysfunction has been associated with persistent bacteremia in hospitalized patients, suggesting that in some situations, treatment that includes administering a quorum sensing inhibitor may be effective to reduce S. aureus invasion prior to BSI. Overall, quorum sensing inhibitors may be an effective tool for combating antibiotic resistance, either alone, as adjuncts to existing antibiotics, or along with potential vaccines or other approaches to augment host defense.

As used herein, the term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Bacterial Strains and Growth Conditions

MRSA strain USA300 LAC (agr-I) was a generous gift from Dr. Frank DeLeo (Rocky Mountain National Laboratories, National Institutes of Health, Hamilton, Mont.). S. aureus strains AH1677 (agr-I), AH430 (agr-II), AH1747 (agr-III) and AH1872 (agr-IV) expressing YFP under the control of the agr::P3 promoter have previously been described (Malone et al., 2009, J Microb Methods 77:251-260). S. epidermidis strain AH3408 (agr-I) expressing sGFP under the control of the agr::P3 promoter has also previously been described (Olson et al., 2014, J. Bacteria 196:3482-3493). Strains AH3469 (AgrC WT) and AH3470 (AgrC R238H) are described below. Unless otherwise noted, bacteria were cultured at 37° C. and 220 rpm with at least a 5:1 air:culture ratio in trypticase soy broth (TSB) (Becton, Dickinson and Company, Sparks, Md.). Early exponential phase bacteria were prepared as described previously (30). Frozen stocks were maintained at −80° C. in TSB supplemented with 10% glycerol. Bacteria were enumerated by serial dilution and plating onto trypticase soy agar containing 5% sheep blood (Becton, Dickinson and Company, Sparks, Md.) followed by overnight incubation at 37° C. The limit for detection of CFUs was 2-log₁₀.

### agr:P3 Promoter Activation Assays

Overnight cultures of S. aureus agr::P3 reporter strains were grown in TSB supplemented with chloramphenicol (Cam) at 10 μg/mL. They were diluted 1:250 into fresh TSB with Cam, and 100 μL aliquots were transferred to 96-well microtiter plates (Costar 3603; Corning, Tewksbury, Mass.) prefilled with 100 μL of media and a two-fold serial dilution series (200 to 0.1 gM) of OHM. OHM was purified from solid phase cultures of Penicillium restrictum as described (Figueroa et al., 2014, J. Nat. Prod 77:1351-1358) and was >95% pure as measured by UPLC (FIG. 7). After mixing in the microtiter plate, the effective dilution was 1:500 and the final OHM concentration ranged from 100 to 0.05 μM, with a final DMSO concentration of 0.1% (v/v) in all wells. Four dilution series were prepared for each reporter, and in addition, four mock DMSO dilution series were included for each reporter strain. Microtiter plates were incubated at 37° C. with shaking (1000 rpm) in a Stuart SI505 incubator (Bibby Scientific, Burlington, N.J.) with a humidified chamber. Fluorescence (top reading, 493 nm excitation, 535 nm emission, gain 60) and OD₆₀₀ readings were recorded at 30-minute increments using a plate reader (Infinite M200; Tecan Systems, San Jose, Calif.).

S. epidermidis AH3408 (agr-L::P3-sGFP) was cultured overnight in TSB supplemented with erythromycin (Erm) at 10 μg/mL. To collect exogenous S. epidermidis AIP1 peptide, spent medium was centrifuged at 3,000×g, passed through a 0.2 μm HT TUFFRYN membrane (Pall, Port Washington, N.Y.), and stored at −20° C. until use. An overnight culture of AH3408 was diluted 1:200 into 500 μL TSB (broth) or TSB with 10% spent medium containing 5 μg/mL OHM or DMSO (vehicle). Cultures were incubated for 24 hours at 37° C., centrifuged, and resuspended in 10% formalin fixative for one minute. Cultures were washed twice by centrifugation and resuspended in PBS. The mean channel fluorescence (MCF) of sGFP was analyzed using an Accuri C6 flow cytometry system (BD Biosciences, San Jose, Calif.). Data were normalized to the broth cultures containing no exogenous AIP1.

### Quantitative PCR

For transcriptional quantification of mouse mRNA, 2.25 cm² sections of skin including and surrounding the abscess were excised, minced and stored in RNAlater (Qiagen, Valencia, Calif.) at −20° C. until use. mRNA was purified using RNeasy kits (Qiagen, Valencia, Calif.) and cDNA was generated using a high-capacity RNA-to-cDNA kit (Applied Biosystems, Foster City, Calif.). Quantitative PCR was performed using an ABI 7900HT RT-PCR system with Taqman Gene Expression Master Mix according to manufacturer's directions (Applied Biosystems, Foster City, Calif.). Predesigned primer and probe sets (Integrated DNA Technologies, Coralville, Iowa) were used for quantitation of mouse il-6, il-1β, tnfα, nlrp3 and hprt. Data are represented as the fold increase relative to hprt as compared to uninfected tissue.

For quantification of S. aureus gene transcription, 500 μL cultures at 2×10⁷ CFU/mL of LAC and/or LACΔagr were grown in TSB at 37° C. with aeration for the indicated times with 50 nM exogenous AIP1 (Biopeptide Co., Inc., San Diego, Calif.) and treatments as indicated. Bacteria were stored at −20° C. in RNAprotect Cell Reagent according to manufacturer's recommendations (Qiagen, Valencia, Calif.) until RNA was purified as previously described (Sully et al., 2014, PLoS Pathog. 10:e1004174). cDNA generation and qPCR was performed as described above for eukaryotic qPCR. Primer and probe sets for quantification of S. aureus genes are listed in Table 2.

Table 2
Oligonucleotides used for S. aureus qPCR
Table S1
Oligonucleotides used for S. aureus qPCR

| Focus | Gene | Oligo | Sequence (5′-3′) | Source |
|---|---|---|---|---|
| Control | 16S | F | TGA TCC TGG CTC AGG ATG A | (1) |
| | | R | TT CGC TCG ACT TGC ATG TA | |
| | | Probe | CGC TGG CGG CGT GCC TA | |
| Virulence | [symbol] | F | ACA ATT TTA GAG AGC CCA ACT GAT | (1) |
| | | R | TCC CCA ATT TTG ATT CAC CAT | |
| | | Probe | AAA AAG TAG GCT GGA AAG TGA TAT TTA ACA | |
| | [symbol] | F | TAT CAA AAG CTT AAT CGA ACA ATT C | (2) |
| | | R | CCC CTT CAA ATA AGA TGT TCA TAT C | |
| | | Probe | AAA GAC CTC CTT TGT TTG TTA TGA AAT CTT ATT TAC CAG | |
| | RNAIII | F | AAT TAG CAA GTG AGT AAC ATT TGC TAG T | (1) |
| | | R | GAT GTT GTT TAC GAT AGC TTA CAT GC | |
| | | Probe | AGT TAG TTT CCT TGG ACT CAG TGC TAT GTA TTT TTC TT | |
| | saeR | F | TGC CAA AAC ACA AGA ACA TGA TAC | (3) |
| | | R | CTT GGA CTA AAT GGT TTT TTG ACA TAG T | |
| | | Probe | TTT ACG CCT TAA CTT TAG GTG CAG AT | |
| | [symbol] | F | ACG GAA AAA CCA GTT CAT GC | (1) |
| | | R | GCT TAT CTT TGC CAA TTA AAG CA | |
| | | Probe | CAG GTT ATA TCA GTT TCA TTC AAC CA | |
| | [symbol] | F | GAT GGT AAC GGA GTA CAT GTC GTT | (4) |
| | | R | TTG CTG GTT GCT TCT TAT CAA CA | |
| | | Probe | ACA TTG CAA AAG CAA ACG GCA CTA CTG C | |
| Stress | asp23 | F | GTT AAC GAC CTT TCA TGT CTA AGA TAC | This |
| response | | R | AAA TTA ACT TTC TCT GAT GAA GTT GTT GA | Study |
| | | Probe | CTT CAC GTG CAG CGA TAC CAG CAA TTT | |
| | crtM | F | GTT TGA AAC GGA CC TGA ATT A | This |
| | | R | ACC AAG TCT TCT TGC GAC AT | Study |
| | | Probe | TGG TGT TGC TGG TAC AGT AGG | |
| | clpB | F | TGG TGT GCG TAT TCA AGA TAG AG | This |
| | | R | GCA CAT GCT TGG TCA ACT AAA T | Study |
| | | Probe | ATC AGA CAA TTC AGC GGC AGC AAC | |
| Meta- | atpG | F | GCC ACT GAT AAT GCA ACT GAA C | This |
| bolism | | R | GCG GAA CCA CCA ACA ATT TC | Study |
| | | Probe | AGA GCG AGA CAA GCA GAA ATT ACG CA | |
| | murQ | F | CTG GTG GAC AAG ATG CTA TGA | This |
| | | R | CAC TCG CGG CAA TTC CTA TAA | Study |
| | | Probe | ATG GCT GTA GAA GGT GCG GAA GAT | |
| | sdhA | F | GCA TTA ATG GTG CG TCA ATA C | This |
| | | R | CTG CCT CTG TCA TCG CTT TA | Study |
| | | Probe | ATG GG GCG ATT TCC TTG CAA ACC | |
| [symbol] / | [symbol] | F | CGA GAG TGA TTG GTG GTA TGA G | This |
| anti- | | R | TCG CTG ACA TGT AGC CAA AG | Study |
| biotic | | Probe | TGC TGG TAT GGT AAT GCC TGG TGT | |
| resis- | mdrA | F | AGT CCT GCA TCT GGA CAA ATT A | This |
| tance | (SAUSA300_ | R | CTT TCG TTT CGC CAT CTT GAC | Study |
| | 2299) | Probe | ACA AGG TGA CAA ACT CGA TAA AGG TGA CA | |
| | NaMDR | F | TGG GAT TAT GTG AAG GTG TTG TA | This |
| | (SAUSA300_ | R | ACG CCG ATA GAC ATG ATA ACT G | Study |
| | 0335) | Probe | ACG TCT TTC ATA CGG CCT TTA TTT GCC | |

(1)Sully et al., 2014, PLoS Pathog. 10:e1004174.
(2)Queck et al., 2008, Mol. Cell 32:150-158.
(3)Voyich et al., 2009, J Infect Dis. 199:1698-1706.
(4)Loughman et al., 2009, J Infect Dis. 199:294-301. [symbol]
indicates data missing or illegible when filed

### Rabbit Red Blood Cell Lysis Assay

The assay was performed as previously described (Bernheimer et al., 1988, Methods Enzymol. 165:213-217). Briefly, LAC was cultured in 5 mL TSB for eight hours with the indicated treatments, centrifuged, and supernatants were filtered through a 0.2 μm HT TUFFRYN membrane (Pall, Port Washington, N.Y.). Serial two-fold dilutions of the supernatant were incubated at 37° C. for one hour in a 4% solution of rabbit red blood cells (rRBCs). Lysis was assessed spectrophotometrically at OD₄₅₀. Data were analyzed by non-linear regression fit to a four-parameter logistic curve and represented as the HA₅₀, which equals 1/dilution required for 50% of complete lysis.

### AgrC Constitutive Reporter Assay

The agrBDCA operon was amplified from strain LAC using primers AgrB+RBS 5′KpnI (GTTGGTACCCAGTGAGGAGAGTGGTGTAAAATTG; SEQ ID NO:1) and AgrA 3′SacI (GTTGAGCTCCTTATTATATTTTTTTAACGTT′ICTCACCGATG; SEQ ID NO:2) and ligated into pRMC2 (Corrigan et al., 2009, Plasmid. 61:126-129). To make a variant with constitutive AgrC activity, we chose the AgrC R238H mutation, which was previously shown to have similar activity in the presence and absence of AIP2 inhibitor and maximal activity in the absence of AIP1 (Geisinger et al., 2009, Proc. Natl. Acad. Sci, USA 106:1216-1221). The AgrC R238H variant was generated by the QuikChange (Agilent Technologies, Santa Clara, Calif.) site-directed mutagenesis method, using primers AgrC R238H fwd (CAACGAAATGCGCAAGTTCCATCATGATTATGTCAATATC; SEQ ID NO:3) and AgrC R238H rev (GATATTGACATAATCATGATGGAACTTGCGCATTTCGTTG; SEQ ID NO:4). To build a destination strain for assessing alpha-hemolysin production, we selected an agrC transposon mutant (NE873) from the Nebraska Transposon Mutant Library (Fey et al., 2013, mBio. 4:e00537-00512) and integrated the pLL29 plasmid at the phage 11 attachment site to confer tetracycline resistance (Luong et al., 2007, Microbiol. Methods. 70:186-190). The above described pRMC2 constructs were transformed into this strain to make reporters AH3469 (AgrC WT) and AH3470 (AgrC R238H). To test OHM, AH3469 and AH3470 were grown overnight with Cam at 10 μg/mL and were diluted 1:500 into 5 mL fresh media with Cam at 10 μg/mL and anhydrotetracycline at 0.025 μg/mL. AIP2 control, OHM, or DMSO (vehicle) were added to each strain at the concentrations indicated. Cultures were grown at 37° C. and 220 rpms for 6.5 hours. Bacteria were pelleted by centrifugation and the alpha-hemolysin containing supernatants were passed through a 0.2 μm HT TUFFRYN membrane (Pall, Port Washington, N.Y.). Rabbit red blood cell lysis assays were conducted as above but with an rRBC concentration of 1% and 25% supernatant (vol/vol) to yield complete lysis. Values are presented as the mean relative lysis compared to vehicle treatment.

### Eukaryotic Cytotoxicity

A549, HEK293, or HepG2 cells were seeded in a 96-well tissue culture plate at 2.5×10⁴ cells per well and incubated at 37° C. with 5% CO₂. 2,3-Bis(2-methoxy-4-nitro-5-sulfophenyl)-2H-tetrazolium-5-carboxanilide (XTT; Sigma-Aldrich, St. Louis, Mo.) and phenazine methosulfate (PMS; Sigma-Aldrich, St. Louis, Mo.) were used to perform an XTT assay as previously described (Scuderio et al., 1988, Cancer Res. 48:4827-4833). After 24 hours, spent media was removed and fresh media containing the indicated drug concentrations or vehicle was added to the cells and incubated for an additional 24 hours. To avoid potential interference with absorbance readings due to the red color of OHM, drug-containing media was replaced with 100 μL of 0.3 mg/mL XTT with 0.015 mg/mL PMS in HBSS and incubated for one hour. Cell viability was assessed by the metabolic reduction of tetrazolium measured at OD₄₉₀. Data are presented as percent viable cells compared to vehicle control.

### EMSA and Flow Cytometry-Based AgrAc Promoter Binding Assays

E. coli expressing the AgrA C-terminal DNA binding domain (AgrA_{C}) along with a 6×-histidine tag was generously provided by Dr. Chuan He (University of Chicago, Chicago, Ill.) and purified as previously described (Sun et al., 2012, Proc. Natl. Acad. Set USA 109:9095-9100). Electrophoretic mobility shift assays (EMSA) were performed as previously described (Sully et al., 2014, PLoS Pathog. 10:e1004174) with purified AgrAc and agr P2 promoter, a 16 base pair duplex DNA probe with a 3′ 6-fluorescein (P2-FAM; Integrated DNA Technologies, Coralville, Iowa)). The duplex DNA contained the high affinity LytTR binding site located in both agr P2 and P3 promoters (Sidote et al., 2008, Structure. 16:727-735). Briefly, 2 μM AgrA_{C} was incubated for 10 minutes at room temperature (RT) with vehicle or the indicated concentrations of OHM in Tris-acetate-EDTA (TAE) buffer with 10 mM dithiothreitol (DTT). Next, 20 ng of P2-FAM DNA probe was added and incubated for an additional 10 minutes. Reactions were loaded onto a 10% PAGE gel and run at 50V in the dark for 20 minutes. DNA migration was assessed by imaging on a FluorChem R (ProteinSimple, Santa Clara, Calif.).

For the flow-based AgrA_{C} promoter binding assays, AgrA_{C} was biotinylated (AgrA_{C}-BTN) using a Thermo Scientific EZ-Link Sulfo-NHS-LC-Biotin kit (Thermo Scientific, Rockford, Ill.) according to manufacturer's directions. AgrA_{C}-BTN was immobilized on 1 μm diameter Dynabeads MyOne Streptavidin T1 beads (Life Technologies, Grand Island, N.Y.) (AgrA_{C}-SA) and beads were suspended in PBS. DNA probe (P2-FAM) was added at 1.6 μM final concentration, along with equimolar competing unlabeled P2, vehicle control or OHM at the indicated concentrations. OHM mediated inhibition of AgrA_{C}-SA binding to DNA probe was measured as decreased mean channel fluorescence (MCF) compared to vehicle control using an Accuri C6 flow cytometry system (BD Biosciences, San Jose, Calif.).

In silico Docking on AgrA_{C}

In silico docking calculations were performed using the Macintosh binary executable of Autodock Vina (Trott et al., 2010, J. Comput. Chem. 31:455-461). OHM was docked onto the B subunit of the AgrA_{C} crystal structure (RSCB Protein Data Bank, accessible on the world wide web at pdb.org, PDB ID 4G4K) (Leonard et al., 2012, Biochemistry. 51:10035-10043; Berman et al., 2000, Nucleic Acids Res. 28:235-242) stripped of heteroatoms. The search box was restricted to the C-terminal region of AgrA_{C}, as described for 9H-xanthene-9-carboxylic acid (Leonard et al., 2012, Biochemistry. 51:10035-10043). Based on initial observations suggesting that OHM bound to the pocket between the side chains of His200, Agr218, Tyr229 and Val232, additional calculations were run in which the size of search box was varied and the side chain torsion angles for different combinations of residues in the region where allowed to be flexible. The reported docking solution was obtained by allowing flexibility in the side chain torsion angles for His200, Agr218, Tyr229 and Val232, and using a search box that was large enough to include both the pocket bounded by the side chains of His200, Agr218, Tyr229 and Val232 and the groove between Val232 and Lys236. Molecular modeling images were prepared using PDB ID 3BS1 and PyMOL (PyMOL Molecular Graphics System, v. 1.5.0.4 Schrodinger, LLC, New York, N.Y.).

### Surface Plasmon Resonance Analysis

To overcome the potential interference for oxidative inactivation of AgrA_{C} during surface plasmon resonance (SPR) analysis, the oxidation-resistant C199S mutation was introduced into the AgrA_{C} expression construct as previously described (Sun et al., 2012, Proc. Natl. Acad Sci. USA 109:9095-9100) using the QuikChange II XL kit (Agilent Technologies, Santa Clara, Calif.). His-tagged AgrA_{C}-C199S was purified as described previously (Sully et al., 2014, PLoS Pathog. 10:e1004174), but without the addition of TCEP or DTT during purification.

SPR binding and kinetics analyses were performed on a Biacore X100 instrument (GE Healthcare, Pittsburgh, Pa.) and evaluated with Biacore X100 Evaluation Software (Version 1.0). His-tagged AgrA_{C}-C199S was immobilized at 10 μg/mL in PBS on an NTA biosensor with the NTA reagent kit (GE Healthcare, Pittsburgh, Pa.). For binding studies, OHM (analyte) was dissolved in running buffer (PBS, 5% DMSO, pH 9), and applied at a flow rate of 30 μL/min with a 180-second contact time and 300-second dissociation time. Data were fit to a 1:1 binding model after subtraction of blank injections and removal of injection spikes from the sensorgrams. NTA biosensor chips were regenerated with the following sequence: two 60-second washes with 350 mM EDTA, a 60-second wash with PBS, a 60-second wash with 500 mM imidazole, followed by a final 60-second wash with PBS. Analyses were performed at 25° C.

### Mouse Model of Skin and Soft Tissue Infection

The mouse model of skin and soft tissue infection was previously described and was implemented with minor modifications (Malachowa et al., 2013, Methods Mol. Biol. 1031:109-116). Early-exponential phase LAC was diluted into USP grade saline (Braun, Irvine, Calif.) to deliver 5-7×10⁷ CFU per mouse. Aliquots of OHM were diluted in 0.5% Hydroxypropylmethylcellulose (HPMC), pH 11, to deliver 0.2 mg/kg per mouse (˜5 μg). Mice were anesthetized with 3% isoflurane at 3 L/min. For prophylactic studies, bacteria and OHM were mixed 1:1 immediately before subcutaneous injection into the right flank in a total volume of 50 μL. For therapeutic studies, bacteria were injected into the right flank in a total volume of 50 μL. Four hours post-infection, OHM or OHM plus antibiotic were injected into the tight flank, dorsal to the infection site, in a total volume of 50 μL. Mice were weighed prior to infection and every day post-infection. Additionally, injection sites were photographed daily to determine abscess and ulcer areas by ImageJ analysis (Schneider et al., 2012, Nal. Methods. 9:671-675). On day three or day seven post-infection, mice were euthanized by CO₂ asphyxiation and a 2.25 cm² section of skin surrounding the abscess was excised. The tissue was mechanically homogenized and serially plated on sheep blood agar to determine bacterial burden. Tissue homogenates were stored at −80° C. until they were rapidly defrosted at 37° C. for cytokine analysis. Day three post-infection homogenate was centrifuged at 12,500×g for 10 minutes and the clarified supernatant analyzed with a custom designed multiplex assay (Merck KGaA, Darmstadt, GER) using a BioPlex 200 with BioPlex Manager software (BioRad Laboratories, Inc., Hercules, Calif.). Abscess tissues collected for H&E staining were fixed overnight in 10% formalin and embedded in paraffin. Five micron sections were then H&E stained and imaged using an Olympus IX70 microscope (Olympus, Center Valley, Pa.).

### Alpha-hemolysin (Hla) Quantification

For detection of Hla in abscess tissue homogenates by western blot, clarified homogenates were rapidly thawed and an aliquot electrophoresed on a 16% Tris-glycine SDS-PAGE gel (Life Technologies, Grand Island, N.Y.) before transfer to a polyvinylidene fluoride membrane. Membranes were blocked for one hour at room temperature, using TBST (20 mM Tris pH 7.5, 150 mM NaCl, 0.1% Tween 20) with 5% non-fat dry milk. Hla was detected using anti-Hla antibody (ab15948; Abeam, Cambridge, Mass.) at 1:1000 and alkaline phosphatase-conjugated secondary antibody. Immunoreactive bands were developed with NBT/BCIP (Thermo Scientific, Rockford, Ill.) and intensity measured using a FluorChem R System and AlphaView software (ProteinSimple, San Jose, Calif.). Relative intensity is the ratio of measured intensity divided by the total protein concentration based on absorbance at 280 nm.

Mouse Mmacrophage Killing of S. aureus

Murine macrophage cells (RAW 264.7) were maintained at 37° C. in 5% CO₂ in high glucose DMEM supplemented with 10% FBS, 2 mM L-glutamine, 10 mM HEPES with 100 U/mL penicillin and 100 μg/mL streptomycin. Twenty-four hours prior to experiments, RAW cells were washed with PBS and media replaced with DMEM as described above, but with 2% FBS without antibiotics. Early exponential phase LAC or LACΔagr were cultured in 3 mL TSB at 2×10⁷ CFU/mL at 37° C. with aeration for five hours with 50 nM exogenous AIP1 (Biopeptide Co., Inc., San Diego, Calif.) and 5 μg/mL OHM or DMSO (vehicle). Bacteria were centrifuged, washed in PBS, sonicated and suspended at 1-2×10⁸ in DMEM, but with 1% FBS without antibiotics. Bacteria were opsonized overnight at 4° C. with rabbit anti-S. aureus IgG at 100 μg/mL (YVS688I; Accurate Chemical & Scientific Co., Westbury, N.Y.). RAW cells were washed with PBS and suspended at 2×10⁷ cells/mL in DMEM with 1% FBS without antibiotics and combined with opsonized bacteria at an MOI of 1:1. Cells were centrifuged at 500×g for three minutes to initiate contact, and incubated at 37° C. in 5% CO₂ for one hour to allow phagocytosis. Lysostaphin (L-0761; Sigma-Aldrich, St. Louis, Mo.) was added at 2 μg/mL for 15 minutes to kill extracellular bacteria and then removed by centrifugation and replacement with fresh media. Half the samples were immediately processed for CFU determination and the other half were incubated for an additional four hours before CFU enumeration. Intracellular bacteria were enumerated by preliminary dilution into PBS/0.1% Triton X-100 followed by sonication and plating onto blood agar.

### Human PMN Assays

PMNs were purified from normal, healthy venous blood as previously described (Nauseef WM, 2014, Methods Mot. Biol. 1124:13-18). Purified PMNs were suspended in HBSS without divalent cations at no more than 3×10⁷ cells/mL and kept on ice until use.

PMN phagosomal killing of S. aureus was conducted as previously described (Pang et al., 2010, J. Innate Immun. 2:546-559), with the following alterations. Prior to opsonization, early exponential phase LAC or LACΔagr were cultured in 3 mL TSB at 2×10⁷ CFU/mL at 37° C. with aeration for five hours with 50 nM exogenous AIP-I (Biopeptide Co., Inc., San Diego, Calif.) and 5 μg/mL OHM or DMSO (vehicle). Bacteria were centrifuged, washed in PBS and opsonized at 5×10⁶ CFU/mL in HBSS with divalent cations supplemented with 20 mM HEPES, 1% HAS, and 10% pooled human serum. Following a 20-minute incubation with tumbling at 37° C., bacteria were pelleted, washed in PBS, and resuspended in HBSS with divalent cations supplemented with 20 mM HEPES. PMNs and opsonized bacteria were combined at an MOI of 1:1 and incubated for 10 minutes at 37° C. Extracellular bacteria were removed by centrifugation at 500×g for five minutes, followed by resuspension of infected PMNs in HBSS with divalent cations supplemented with 20 mM HEPES and 1% HSA. Infected PMNs were incubated at 37° C. for 120 minutes and aliquots were removed at 0, 30, 60 and 120 minutes. Aliquots were diluted into PBS/0.1% Triton X-100 to lyse cells, and then serially diluted and plated on blood agar for CFU enumeration.

Lysis of PMNs by S. aureus supernatant was conducted as previously described, with minor modifications (Sully et al., 2014, PLoS Pathog. 10:e1004174). Briefly, LAC was cultured in 3 mL TSB for five hours with 5 μg/mL OHM or vehicle, centrifuged, and supernatants were filtered through a 0.2 μm HT TUFFRYN membrane (Pall Corp., Port Washington, N.Y.). Supernatants were stored at −80° C. and thawed on ice prior to use. PMNs were washed with PBS and resuspended in RPMI supplemented with 10 mM HEPES and 1% HSA. PMNs at a density of 3×10⁶ cells/mL in 100 μL were added to 100 μL RPMI, RPMI with 10% TSB (vol/vol) or RPMI with 10% S. aureus supematant prepared as above. PMNs were incubated at 37° C. and 5% CO₂ for two hours. Following incubation, supernatants were collected by centrifugation at 3,000×g for 5 min and assessed for LDH release according to manufacturer's specifications (CytoTox 96 Non-radioactive Cytotoxicity assay, Promega Co., Madison, Wis.). Triton X-100 was added at a final concentration of 0.1% (vol/vol) as a 100% lysis control while cell free RPMI with 5% TSB served as a blank. Data are normalized to 100% lysis control.

### Statistical Analysis

Statistical evaluations were performed using GraphPad Prism v.5.04. In vitro data were analyzed by the two-tailed Student's t-test and in vivo data were analyzed by the Mann-Whitney U test for non-parametrics. Results were considered significantly different at p<0.05.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A pharmaceutical composition comprising:
a polyhydroxyanthraquinone, or a pharmaceutically acceptable salt thereof, in an amount effective to inhibit quorum sensing in a microbe; and
a pharmaceutically acceptable carrier.

2. A pharmaceutical composition comprising:
a polyhydroxyanthraquinone, or a pharmaceutically acceptable salt thereof, in an amount effective to antagonize AgrA function in a microbe; and
a pharmaceutically acceptable carrier.

3. A pharmaceutical composition comprising:
a polyhydroxyanthraquinone, or a pharmaceutically acceptable salt thereof, in an amount effective to attenuate a skin and soft tissue infection (SSTI) of a subject by a microbe; and
a pharmaceutically acceptable carrier.

4. A pharmaceutical composition comprising:
a polyhydroxyanthraquinone, or a pharmaceutically acceptable salt thereof, in an amount effective to reduce an inflammatory response in a subject in response to infection by a microbe; and
a pharmaceutically acceptable carrier.

5. The pharmaceutical composition of claim 1 wherein the polyhydroxyanthraquinone comprises ω-hydroxyemodin (OHM).

6. The pharmaceutical composition of claim 1 comprising a combination of two or more polyhydroxyanthraquinones.

7. The pharmaceutical composition of claim 1 further comprising an antibiotic.

8. The pharmaceutical composition of claim 7 wherein the antibiotic comprises a bacteriocidal antibiotic.

9. The pharmaceutical composition of claim 7 wherein the antibiotic comprises a bacteriostatic antibiotic.

10. The pharmaceutical composition of claim 9 wherein the bacteriostatic antibiotic comprises a lincosamide.

11. The pharmaceutical composition of claim 10 wherein the linosamide comprises clindamycin.

12. A method of treating a subject having, or at risk of having, an infection by a microbe, the method comprising:
administering to the subject an amount of a polyhydroxyanthraquinone effective to inhibit quorum sensing by the microbe.

13. A method of treating a subject having, or at risk of having, an infection by a microbe, the method comprising:
administering to the subject an amount of a polyhydroxyanthraquinone effective to antagonize AgrA function in the microbe.

14. A method of treating a subject having, or at risk of having, an infection by a microbe, the method comprising:
administering to the subject an amount of a polyhydroxyanthraquinone effective to attenuate a skin and soft tissue infection (SSTI) of a subject by the microbe.

15. A method of limiting damage to immune cells of a subject by a microbial virulence factor, the method comprising:
administering to the subject an amount of a polyhydroxyanthraquinone effective to limit damage to immune cells of the subject by a microbial virulence factor.

16. The method of claim 15 wherein the immune cells comprise macrophages or polymorphonuclear leukocytes (PMN).

17. A method of limiting damage to a tissue of a subject caused by a microbial virulence factor, the method comprising:
administering to the subject an amount of a polyhydroxyanthraquinone effective to limit damage to the tissue by a microbial virulence factor.

18. A method of treating a subject having, or at risk of having, an infection by a microbe, the method comprising:
administering to the subject an amount of a polyhydroxyanthraquinone effective to reduce, limit progression, ameliorate, or resolve, to any extent, a symptoms or clinical sign of infection by a microbe.

19. The method of claim 18 wherein the infection comprises an infection secondary to diabetes.

20. The method of claim 18 wherein the subject further has, or is at risk of having, diabetes.

21. The method of claim 12 wherein the microbe comprises a pathogen.

22. The method of claim 12 wherein the microbe comprises a member of the family Staphylococcaceae.

23. The method of claim 22 wherein the microbe is Staphylococcus aureus.

24. The method of claim 23 wherein the S. aureus comprises methicillin-resistant S. aureus (MSRA).

25. The method of claim 12 wherein the polyhydroxyanthraquinone is administered to the subject before the subject exhibits a symptom or clinical sign of infection.

26. The method of claim 12 wherein the polyhydroxyanthraquinone is administered to the subject after the subject exhibits a symptom or clinical sign of infection.

27. The method of claim 12 wherein the polyhydroxyanthraquinone comprises ω-hydroxyemodin (OHM) or an analogue thereof.

28. A method for attenuating virulence of a Staphylococcus spp., the method comprising:
contacting the Staphylococcus spp. with an amount of a polyhydroxyanthraquinone effective to attenuate virulence of the Staphylococcus spp.

29. The method of claim 28 wherein the polyhydroxyanthraquinone attenuates production of alpha-hemolysin.

30. The method of claim 28 wherein contacting the polyhydroxyanthraquinone with the Staphylococcus spp. downregulates expression of at least one virulence gene in the Staphylcoccus spp.

31. The method of claim 12 wherein the polyhydroxyanthraquinone is administered to a subject by injection.

32. The method of claim 28 wherein the injection comprises subcutaneous injection.

33. The method of claim 12 wherein the polyhydroxyanthraquinone is administered to a subject by elution from medical dressing.

34. The method of claim 12 wherein the subject is a mammal.

35. The method of claim 34 wherein the mammal is a human.
